(19) United States
(12) Patent Application Publication
(10) Pub. No.: US 2013/0122076 A1
(43) Pub. Date: May 16, 2013

(54) Transdermal Patch Having Ultrasound Transducer for Administering Thrombolytic Reagents to Patients Having a Protein Misfolding Disease

(71) Applicant: Mathew Gelfand, Lido Beach, NY (US)

(72) Inventor: Mathew Gelfand, Lido Beach, NY (US)

(21) Appl. No.: 13/430,881

(22) Filed: Mar. 27, 2012

Related U.S. Application Data
(60) Provisional application No. 61/558,650, filed on Nov. 11, 2011.

(51) Int. Cl. A61K 9/70; A61P 25/28; A61P 9/10; A61P 3/10; A61P 37/00; A61P 25/16; A61K 38/49; A61P 25/00

(52) U.S. Cl. 424/443; 424/94.64

(57) ABSTRACT

The present invention relates to a method and device for treating patients with a Protein Misfolding Disease by chronically administering an effective dose of a thormbolytic reagent over an intermittent period of time. The thrombolytic reagents degrade the misfolded proteins that accumulate and that can become toxic in such patients. The administration is administered transdermally via a transdermal patch that is equipped with an ultrasound transducer for enhancing the penetration of the thrombolytic reagent into the bloodstream of a patient by increasing the permeability of the patient's skin.

This application claims priority on U.S. Provisional Patent Application Ser. No. 61/558,650 filed Nov. 11, 2011.

### FIELD OF THE INVENTION

The present invention relates to delivery systems and methods that provide for the chronic and intermittent transdermal administration of low doses of thrombolytic (fibrinolytic) reagents over a prolonged period of time, such as tissue plasminogen activator (t-PA), streptokinase and/or urokinase, for the treatment of patients suffering from a Protein Misfolding Diseases which include Alzheimer's disease, Atherosclerosis, Diabetes, Parkinson's disease, Mad Cow Disease and conditions of Multiple Sclerosis and Cystic Fibrosis. More specifically, the present invention relates to the transdermal administration of the thrombolytic (fibrinolytic) reagents through a dermal patch where the dermal patch contains an ultrasound transducer to enhance the transdermal delivery of the thrombolytic (fibrinolytic) reagents.

### BACKGROUND OF THE INVENTION

### Tissue Plasminogen Activator

Thrombolytic (fibrinolytic) drugs act on the endogenous fibrinolytic system by converting plasminogen to the potent proteolytic enzyme plasmin. Plasmin in turn degrades fibrin clots and other plasma proteins. A number of thrombolytic (fibrinolytic) drugs, including urokinase, streptokinase and t-PA, are currently used to treat acute vascular disease.

Tissue plasminogen activator (t-PA) activates plasminogen to generate the proteinase plasmin which plays an important role in the degradation of fibrin. t-PA has been a particularly important pharmaceutical agent for use in treatment of vascular diseases due to its ability to dissolve blood clots in vivo. FIG. 3 provides a flow chart of the process for repairing an injury to a blood vessel. After a blood vessel injury occurs, a plasma protein called thromboplastin aids in blood coagulation by converting prothrombin to thrombin. Next, C-reactive protein (CRP) is stimulated by the tissue injury and in turn stimulates plasminogen activator inhibitor-1 (PAI-1) which stimulates t-PA. The t-PA activates plasminogen to generate plasmin which degrades the fibrin clot. Once the clot is dissolved, the extracellular matrix scaffold for tissue reconstruction begins the healing process. The foregoing description highlights the important connection that t-PA has throughout the whole blood vessel injury reconstruction process, including at the start of blood coagulation.

It has also been noted that t-PA degrades beta-amyloid plaques by activating plasminogen. (Wang et al., Beta-Amyloid Degradation and Alzheimer's Disease, J. Biomedicine and Biotech., 2006: 1-12 (2006)). t-PA was originally identified and purified from natural sources. Through the use of recombinant DNA techniques, DNA clones encoding the t-PA molecule have been identified and characterized leading to a determination of the DNA sequence and deduced amino acid sequence of t-PA (U.S. Pat. No. 4,853,330).

Several variants of t-PA have also been developed that address some of the disadvantages associated with the use of t-PA. These disadvantages include the short half life and fast clearance rate of t-PA. Such variants include those described in EPO Patent Publication No. 199,574, that have amino acid substitutions at the proteolytic cleavage sites at amino acid positions 275, 276 and 277. These forms are referred to as protease-resistant one-chain t-PA variants in that, unlike natural t-PA, they exist in either one chain or two chain form and are resistant to proteolytic cleavage. Such variants are thought to be superior to natural t-PA for pharmaceutical uses in that they are more stable. In addition, a variety of glycosylation mutants exist at positions 117, 119, 184-186 and 448-450 which exhibit higher specific activity than natural t-PA.

A general review of plasminogen activators and derivatives thereof can be found in Harris (1987, Protein Engineering 1:449-458); Pannekock et al. (1988, Fibrinolysis 2:123-132); and Ross et al. (1988, Annual Reports in Medicinal Chemistry, Vol. 23, Chapter 12), each of which is incorporated by reference herein.

Thrombolytic (fibrinolytic) drug therapy, and in particular, t-PA, has been used for treating acute ischemic stroke. There are two ways that t-PA has been administered—intravenously, or intra-arterially directly at the clot site. In these methods, the t-PA is delivered through the blood vessels to break up the clot that is disrupting the blood flow. Before a dose may be administered there are a number of criteria that first must be considered. First, there must be no evidence of bleeding in potential t-PA recipients because thrombolytic therapy can exacerbate a hemorrhagic stroke. Furthermore, patients taking blood thinners are also not allowed to receive t-PA. In addition, other important considerations include elevated blood pressure or blood sugar, recent surgery, low platelet count, and end-stage liver or kidney disorders. The foregoing therapies are can last for as long as 13 days but are recommended to not exceed a total t-PA administration of 40 mg to 90 mg over the life of the therapy 9 because of the above considerations. As a result of the relatively short amount time of the foregoing therapies, i.e. a maximum therapy life of 13 days, the therapies are not able to sustain a desired level of a thrombolytic (fibrinolytic) reagent and/or fibrinogen and/or fibrin split products in the body of a patient over a prolonged period of time as in the present invention. The present invention, on the other hand, allows for the chronic administration of the thrombolytic (fibrinolytic) drugs over an intermittent schedule where the intermittent schedule can last for at least one month. FIG. 1 represents a flow chart that shows the known relationship between elevated cholesterol, blood cot formation and tissue plasminogen activator.

### Protein Misfolding Disease

Protein Misfolding Disease or proteopathy refers to a class of diseases in which certain proteins become structurally abnormal and as a result disrupt the function of cells, tissues and organs of the body. The proteins in this class of disease fail to fold into their normal configuration. In this misfolded state the proteins can become toxic or they can lose their normal function. Several neurodegenerative and other diseases are believed to result from the accumulation of amyloid fibrills formed by misfolded proteins. Diseases classified as Protein Misfolding Diseases include but are not limited to Alzheimer's disease, Parkinson's disease, Atherosclerosis, Diabetes mellitus, Mad Cow Disease, conditions of Multiple Sclerosis and Cystic Fibrosis, Primary systemic amyloidosis, Ig heavy-chain-associated amyloidosis, Secondary systemic amyloidosis, Senile systemic amyloidosis, Hemodialysis-related amyloidosis, Hereditary systemic ApoAI amyloidosis, Hereditary systemic ApoAII amyloidosis, Finnish hereditary amyloidosis, Hereditary lysozyme amyloidosis, Hereditary cystatin C amyloid angiopathy, Injection localized amyloidosis, Hereditary renal amyloidosis, Senile seminal vesicle amyloid, Familial subepithelial corneal amyloidosis, Cataract, Medullary thyroid carcinoma, Lewy-body dementia, Huntington's disease, Spongiform encephalopathies, Hereditary cerebral hemorrhage with amyloidosis, Amyotrophic lateral sclerosis, Familial British dementia, Familial Danish dementia, Familial amyloidotic polyneuropathy, Frontotemporal dementias, and Sickle cell anemia.

### Alzheimer's Disease

Alzheimer's disease is a type of Protein Misfolding Disease that causes problems with memory, thinking, and behavior. It is a progressive disease where symptoms gradually worsen over a number of years through the damaging and killing of brain nerve cells, i.e., neurons. Two suspected causes of the damaging and killing of neurons are plaques and tangles. Plaques are deposits of a protein fragment called beta-amyloid that build up in the spaces between nerve cells. The amyloid plaques are composed of a tangle of regularly ordered amyloid fibrillar aggregates that are characteristic of a Protein Misfolding Disease. Tangles are twisted fibers of another protein called tau that build up inside cells.

Most people develop plaques and tangles as they age; however, those diagnosed with Alzheimer's disease tend to develop substantially more. In addition, they also tend to develop in similar patterns, beginning in areas important for memory before spreading to other regions. It is not yet known what role plaques and tangles play in Alzheimer's disease, but it is believed that they play a role in blocking nerve cell communication and disrupting processes that cells need to survive. This leads to the damage and destruction of the neurons.

Patients diagnosed with Alzheimer's disease live an average of 8 years after their symptoms become noticeable but survival can range from 4 to 20 years. There is no current cure for Alzheimer's but treatments are administered that delay the progression of the disease.

The present invention is directed to a device and method for extinguishing the misfolded proteins that accumulate in patients with a type of Protein Misfolding Disease and specifically the beta-amyloid plaques that accumulate in a patient with Alzheimer's or other protein misfolding disease by the administration of a thrombolytic (fibrinolytic) reagent, such as t-PA. In the present invention, the t-PA is administered intermittently at low doses over a long period of time to maintain serum concentrations between about 0.1 mg and 50 mgs. The intermittent period may be every day, once a week, once a month, twice a week, twice a month, three times a week, three times a month, etc. The period of time that the therapy may last can be measured weekly and can be more than, less than or equal to a week, including two weeks, three weeks, four weeks, etc.; the period may be measured monthly and can be more than, less than, or equal to a month, including two months, three months, four months, five months, six months, etc.; or the period may be measured yearly and can be more than, less than or equal to a year, such as a year and a half, two years, two and a half years, three years, etc. In addition, the present invention is also directed to an anticoagulant therapy for mitigating the damaging effects that blood clots may have in patients with a type of Protein Misfolding Disease, particularly Alzheimer's disease.

### OBJECTS OF THE INVENTION

It is an object of the invention to provide an improved transdermal delivery system and method for treating patients afflicted with a Protein Misfolding Disease by chronically and intermittently administering a low dose of thrombolytic (fibrinolytic) reagents.

It is another object of the invention to provide an improved transdermal delivery system and method that degrades the amyloid plaques and other misfolded proteins that form in patients suffering from a Protein Misfolding Disease.

It is yet another object of the invention to provide an improved transdermal delivery device that includes a transdermal patch that has an ultrasound transducer for providing enhanced delivery of one or more thrombolytic (fibrinolytic) reagents to a Alzheimer's patient's bloodstream.

It is still another object of the invention to provide an improved delivery system of thrombolytic (fibrinolytic) reagents that extinguishes the beta-amyloid plaques that accumulate in the brain of a patient afflicted with Alzheimer's disease.

It is a further object of the invention to provide an improved transdermal delivery method that provides an enhanced administration of a thrombolytic (fibrinolytic) reagent through the use of a transdermal patch that has an ultrasound transducer.

It is an even further object of the invention to provide a therapeutic method for treating patients with Alzheimer's disease or another Protein Misfolding disease by the intermittent administration of therapeutically effective doses of a thrombolytic (fibrinolytic) reagent.

### SUMMARY OF THE INVENTION

The present invention relates to a method and apparatus for treating patients with a Protein Misfolding Disease, and in particular Alzheimer's disease, by the chronic and intermittent transdermal administration of low doses of thrombolytic (fibrinolytic) reagents such as tissue plasminogen activator (t-PA), streptokinase and/or urokinase. The transdermal administration is preferably via a dermal patch that is equipped with an enhancing agent, such as an ultrasound transducer, for increasing the permeability of the skin and thus the penetration of the reagents into a patient's bloodstream.

The thrombolytic (fibrinolytic) reagent should be administered in a way that is sufficient to achieve serum concentrations of between about 0.1 and 50 mgs. Therefore, an object of the invention is to provide a dose-controlling transdermal applicator for thrombolytic (fibrinolytic) compositions such as t-PA.

The present invention may be used to treat patients suffering from a Protein Misfolding Disease, such as Alzheimer's disease, Atherosclerosis, Diabetes mellitus, Parkinson's disease, Mad Cow Disease, and conditions of Multiple Sclerosis and Cystic Fibrosis by degrading the amyloid plaques and/or other misfolded proteins that have accumulated. Other protein misfolding diseases that the present invention may treat include but are not limited to Primary Systemic Amyloidosis, Ig heavy-chain-associated amyloidosis, Secondary systemic amyloidosis, Senile systemic amyloidosis, Hemodialysis-related amyloidosis, Hereditary systemic ApoAI amyloidosis, Hereditary systemic ApoAII amyloidosis, Finnish hereditary amyloidosis, Hereditary lysozyme amyloidosis, Hereditary cystatin C amyloid angiopathy, Injection localized amyloidosis, Hereditary renal amyloidosis, Senile seminal vesicle amyloid, Familial subepithelial corneal amyloidosis, Cataract, Medullary thyroid carcinoma, Lewy-body dementia, Huntington's disease, Spongiform encephalopathies, Hereditary cerebral hemorrhage with amyloidosis, Amyotrophic lateral sclerosis, Familial British dementia, Familial Danish dementia, Familial amyloidotic polyneuropathy, Frontotemporal dementias, Sickle cell anemia, Spinocerebral ataxia type 6, Fabry's disease, Spinobulbular muscular atrophy, Ataxia, Wilson disease, Atrial amyloidosis of heart, Dentatorubral pallidoluysian atrophy, Hereditary cerebral amyloid angiopathy, Gaucher's disease, Medullary carcinoma of thyroid, Cystic fibrosis, Marfan syndrome, Fragile X syndrome, Fragile XE syndrome, Alexander disease, Light chain amyloidosis, Fatak systemic amyloidosis, Machado-Joseph disease, Hereditary systemic amyloidosis, Myotonic dystrophy, Aquaporin-Vasopressin-1, Cancer, Prion disease, and Retinitis pigmentosa protein. The proteins associated with protein misfolding diseases include β-amyloid, Tau, Modified low-density lipoprotein, Islet amyloid polypeptide, Amylin, α-Synuclein, Immunoglobulin light chain, Immunoglobulin heavy chain, Serum amyloid, Transthyrein, β₂-Microglobulin, apolipoprotein A-I, apolipoprotein A-II, Gelsolin, Lysozyme, Cystatin G, Insulin, Fibrinogen, Lactoferrin, Seminogelin, Crystallin, Calcitonin, Huntington, Prion, Cystatin C, Superoxide dismutase, Abri, ADan, Hemoglobin, α_{1A}-voltage-dependent calcium channel subunit, α-Galactosidase A, Androgen receptor, Ataxins, ATP7B, Atrial natriuretic factor, Atrophin, β-Glucocerebrosidase, Cystic fibrosis transmembrane regulator protein, Fibrillin, Fragile X mental retardation-1 protein, Fragile X mental retardation-2 protein, Glial fibrillary acidic protein, Gonadotropin-release hormone receptor, Ig V_{L} domain, Machado-Joseph disease protein 1, Medin, Myotonic dystrophy protein kinase, Nephrogenic diabetes insipidus, p53, Rhodopsin and von Hippel Lindau protein.

The present invention, in particular, may be used to treat a person afflicted with Alzheimer's disease by degrading the beta-amyloid plaques that have formed in the patient's brain.

The delivery system of the present invention is preferably directed to a dermal patch that contains one or more thrombolytic (fibrinolytic) reagents and an ultrasound transducer to enhance the effects of the administration. The dermal patch is configured to provide the sustained release of the thrombolytic (fibrinolytic) reagent over a prolonged period of time during the administration. The ultrasound transducer increases the permeability of the skin to allow for greater penetration of the reagents.

It is preferred that the dermal patch be applied to a patient to provide the sustained release of a therapeutic amount of the thrombolytic (fibrinolytic) reagents into a patient's bloodstream during an administration event. The administration event is preferably over a prolonged period of time and the sustained release is such that the serum concentration levels discussed herein are achieved. In another embodiment of the invention one or more thrombolytic (fibrinolytic) reagents may be combined with slow release gel formulations which may be applied topically to the patient. In this embodiment an ultrasound may be applied to the slow release gel formulations to increase the permeability of the skin and allow for greater penetration of the reagent. In an alternate embodiment, the t-PA may be administered intravenously or in any other manner that is used in the art, such as but not limited to a microchip that may be implanted in a patient.

The present invention may also be used to treat plaque that accumulates on the teeth of a patient. In this embodiment, the t-PA may be topically applied in a gel or other appropriate form to one or more teeth of a patient to eliminate and/or reduce the accumulation of the plaque. The t-PA in this embodiment may be placed on any surface and/or near any region of the teeth where plaque is accumulated or where it is desirable to prevent the accumulation of plaque. In particular, one region where the t-PA may be applied to the teeth is at or near the gum line of a patient. In this particular embodiment, the t-PA may be applied as such so that it contacts the teeth and gums to ensure that the plaque is adequately reached by the t-PA, including plaque that accumulates below the gum line. By placing the t-PA so that it overlaps the teeth and gums, the t-PA can penetrate into the seam between the teeth and gums so that it reaches plaque below the gum line. The amount of t-PA that may be applied and the intermittency of the application may include that which has been disclosed herein for the transdermal application of t-PA.

### BRIEF DESCRIPTION OF THE DRAWINGS

FIG. 1 is a flow chart that shows the relationship between cholesterol and blood clotting in a person's body.

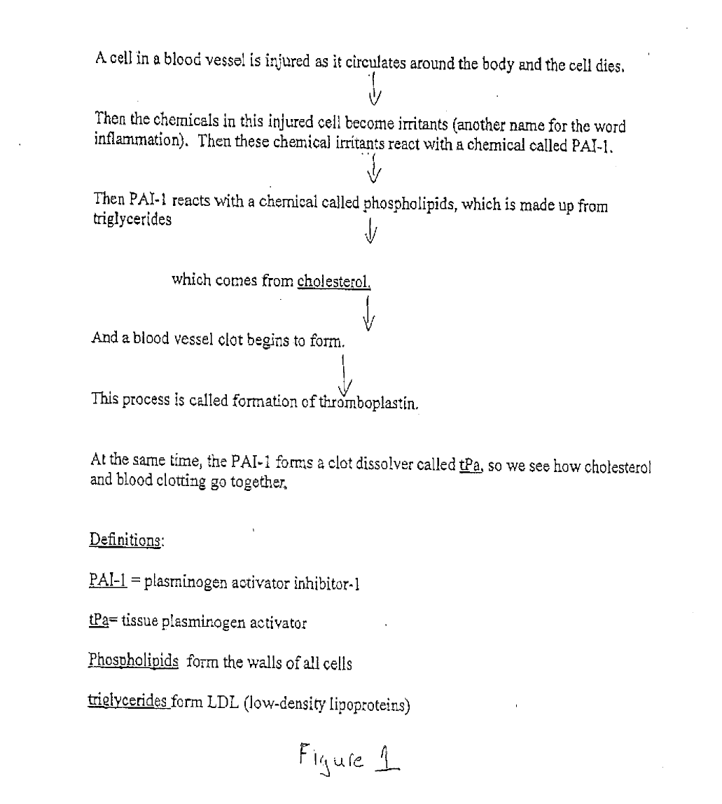

FIG. 2 is a graphical representation of the relationship between the amount of t-PA to be administered and the frequency of t-PA administration.

FIG. 3 is a flow chart that shows the process for repairing an injury to a blood vessel.

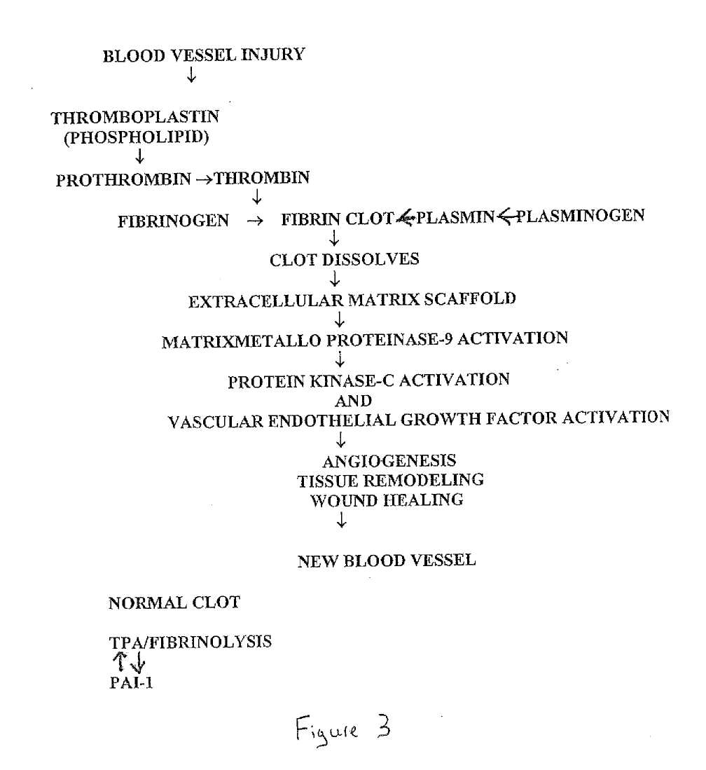

### DETAILED DESCRIPTION OF THE INVENTION

The present invention is directed to an improved delivery system and method for treating patients afflicted with a Protein Misfolding Disease, such as but not limited to Alzheimer's disease, Parkinson's disease, Atherosclerosis, Diabetes, Mad Cow Disease and conditions of Multiple Sclerosis and Cystic Fibrosis. More specifically, the invention relates to the chronic and intermittent transdermal administration of low doses of thrombolytic (fibrinolytic) reagents, such as t-PA, to degrade the misfolded proteins that have accumulated. In particular, the present invention can be used to treat patients suffering from Alzheimer's disease by degrading the beta-amyloid plaques that have accumulated in the brain.

The improved delivery system of the present invention is preferably a transdermal delivery system that delivers thrombolytic (fibrinolytic) reagents, such as t-PA, into the bloodstream of a patient with the aid of an enhancing agent. The transdermal drug delivery system is preferably a dermal patch that contains the t-PA to be administered to a human patient where the t-PA is mixed with suitable carriers or excipient(s) at doses therapeutically effective to degrade amyloid plaques or prevent other vascular conditions. The denial patch includes an enhancing agent in the form of an ultrasound transducer for increasing the pore size of the skin for greater penetration of the thrombolytic (fibrinolytic) reagent into a patient's bloodstream.

The thrombolytic (fibrinolytic) reagents may be administered daily, weekly, bi-weekly, monthly or yearly. The desired goal of any such delivery system is a constant long term delivery of thrombolytic (fibrinolytic) reagents. Such thrombolytic (fibrinolytic) reagents include, for example, t-PA, streptokinase and urokinase, etc.

### Thrombolytic (Fibrinolytic) Reagents

The thrombolytic (fibrinolytic) reagents to be used in the practice of the invention, herein defined as any reagents which have fibrinolytic activity, may be derived from a variety of different sources. For example, the t-PA may be produced in large quantities using recombinant DNA techniques well known to those skilled in the art such as those disclosed in U.S. Pat. No. 4,853,330 which is incorporated herein by reference. Alternatively, the t-PA may be obtained from a number of commercially available sources such as but not limited to Activase® and TNKase® supplied by Genentech, Inc. and isomers thereof.

When using t-PA, it is within the scope of the invention that variants of naturally occurring t-PA may also be used in the practice of the invention. In preferred embodiments, such variants of t-PA may have an increased half life or a slower rate of clearance from the body. For example, variants having amino acid substitutions at the proteolytic cleavage sites at position 275, 276 and 277 which render t-PA preparations more stable may be used. Glycosylation mutants at amino acids 117-119, 184-186 and 448-45 exhibit a higher specific activity and such variant may also be used in the practice of the invention. t-PA can also be modified to delete amino acids 51-87 which results in a variant having a slower clearance from plasma. These variants represent only a subset of the known variants of t-PA which may be used in the presently claimed delivery systems.

It is also within the scope of the present invention that thrombolytic (fibrinolytic) reagents other than t-PA may be used in the practice of the invention. Such agents include urokinase and streptokinase, both of which may be obtained from commercial sources (Urokinase, Abbott Laboratories; Streptokinase, Pharmacia Adria).

Method of Treating Patients with a Protein Misfolding Disease

The present invention relates to methods of treating patients with a Protein Misfolding Disease, such as but not limited to Alzheimer's disease, Parkinson's disease, Atherosclerosis, Diabetes, Mad Cow Disease and conditions of Multiple Sclerosis and Cystic Fibrosis, by the chronic and intermittent administration of low doses of thrombolytic (fibrinolytic) reagents. In particular, the present invention may be used to degrade amyloid plaques that accumulate in Alzheimer's patients and that can become toxic and result in a loss of brain function if not treated.

Pharmaceutical compositions suitable for use in the present invention include compositions wherein the thrombolytic (fibrinolytic) ingredients are contained in an effective amount to achieve its intended purpose. More specifically, a therapeutically effective amount means an amount effective to degrade amyloid plaques or other misfolded proteins in the subject being treated. A therapeutically effective dose refers to that amount of the compound that results in plasma levels of the thrombolytic (fibrinolytic) reagent which are sufficient to maintain the beneficial therapeutic effects.

The amount of composition administered will, of course, be dependent on the subject being treated, on the subject's weight, the severity of the affliction, the manner of administration and the judgment of the prescribing physician. It should be noted that the attending physician would know how to and when to terminate, interrupt or adjust therapy to lower dosage due to toxicity. Conversely, the attending physician would also know to adjust treatment to higher levels if the clinical response is not adequate (precluding toxicity).

In administering thrombolytic (fibrinolytic) reagents to the patient, it is particularly important to monitor the patient for excessive bleeding or tendencies to bleed. A variety of different diagnostic tests, which are well known to those skilled in the art, may be used to access the patient's susceptibility to bleeding due to administration of the thrombolytic (fibrinolytic) reagents. Such assays include a complete blood count (CBC), or a determination of prothrombin or partial prothrombin time. It is preferred that a D-dimer test is performed before a dose of t-PA is administered.

The magnitude of a dose of the t-PA will vary with the patient to be treated. Again, it should be noted that the clinician or physician would know when to interrupt and/or adjust the treatment dose due to toxicity. The dose, and perhaps the dosage frequency, will also vary according to the age, body weight, and response of the individual patient.

In general, a total daily dose range of t-PA should be sufficient to achieve serum concentration levels ranging between about 0.1 and 50 mgs. For smaller patients (less than 65 kg), a dose that is on the lower end of the aforementioned range t-PA range should be administered. In addition, it is further recommended that patients over 65 years, and those with impaired renal, or hepatic function, initially receive low doses, and that they be titrated based on individual clinical response(s) and blood level(s).

### Thrombolytic (Fibrinolytic) Transdermal Drug Delivery System

The present invention also relates to a transdermal drug delivery system for transdermally delivering a quantity of thrombolytic (fibrinolytic) reagents, including t-PA, into the bloodstream of patients suffering from a Protein Misfolding Disease, such as but not limited to Alzheimer's disease, Parkinson's disease, Atherosclerosis, Diabetes, Mad Cow Disease and conditions of Multiple Sclerosis and Cystic Fibrosis. The transdermal drug delivery system is preferably a dermal patch that contains the t-PA to be administered to a patient where the t-PA is mixed with suitable carriers or excipient(s) at doses therapeutically effective to treat a patient with a Protein Misfolding Disease. The dermal patch contains an administration enhancing agent in the form of an ultrasound transducer for increasing the pore size of a patient's skin for greater penetration of the thrombolytic (fibrinolytic) reagent into the bloodstream.

The formulations of the present invention normally will consist of t-PA with a carrier, or diluted by a carrier. Some examples of the diluents or carriers which may be employed in the pharmaceutical compositions of the present invention are lactose, dextrose, sucrose, sorbitol, mannitol, propylene glycol, liquid paraffin, white soft paraffin, kaolin, microcrystalline cellulose, calcium silicate, silica polyvinylpyrrolidone, cetostearyl alcohol, starch, gum acacia, calcium phosphate, cocoa butter, oil of theobroma, arachis oil, alginates, tragacanth, gelatin, syrup B.P., methyl cellulose, polyoxyethylene sorbitan monolaurate, ethyl lactate and propylhydroxybenzoate, sorbitan trioleate, sorbitan sesquioleate and oleyl alcohol.

Because of the short shelf life of t-PA in solution, formulations of t-PA in aqueous solutions, gels, etc. are stored under refrigeration to preserve the activity of the t-PA. Lyophilized preparations of t-PA may be stored at room temperature and protected from excessive exposure to light without loss of activity.

The transdermal delivery of t-PA in accordance with the present invention can be designed so that the rate of delivery of the t-PA closely follows the rate of clearance of the t-PA from the patient's body, thus keeping constant levels of the t-PA in the blood, thereby reducing t-PA waste and overdosing. The use of such a drug delivery system also provides a comfortable, convenient non-invasive method for unattended delivery of t-PA over a prolonged time period.

The transdermal delivery system is preferably in the form of a transdermal patch that is equipped with an ultrasound transducer. The patch preferably consists of an ultrasound transducer and a reservoir of drug material located behind a rate controlling membrane. The patch is impregnated with the t-PA and placed on the skin of the patient, and the ultrasound transducer activated to increase skin permeability and allow the drug to penetrate readily into the body.

The transdermal patch is prepared to contain a solution of t-PA. The t-PA is dispersed in the solution, suspension or gel in a dissolved or undissolved state. The drug reservoir of the patch containing a solution, suspension or gel of t-PA also includes an ultrasound transducer to increase the skin's permeability. Other permeation enhancers may also be included in the dermal patch to increase the skin penetration of the t-PA. Such permeation enhancers include those described in U.S. Pat. No. 4,573,966, which is incorporated by reference herein. Permeation enhancers may include plasticizer type enhancers such as lower alky and alkoxy esters of pharmaceutically acceptable fatty acids, fatty acid esters, fatty alcohols and similar hydrophobic compounds that are capable of increasing the permeability of drugs to the skin. In addition, solvent type enhancers may be used to increase the delivery of drugs through the skin. Such enhancers generally refer to relatively hydrophilic compounds having molecular weights of less than 200. More preferably, solvent type enhancers have a molecular weight of less than 150. They are also generally greater than 2 wt % soluble in water, and are preferably greater than 10 wt % soluble in water. Typically, solvent type enhancers include pharmaceutically acceptable lower alkyl alcohol, aryl alcohol, or polyol, for example, ethanol, propanol, butanol, benzyl alcohol, glycerin, or propylene glycol. as well as diluents, such as water or other additives. The solution of t-PA may be formulated to include vascular permeability factors (VPFs), as described in U.S. Pat. No. 5,503,843, which cause a rapid and reversible increase in blood vessel permeability. Such VPF may be added to the t-PA solution to facilitate the uptake of t-PA into the blood vessels of the skin. In addition, gelling agents may be added to increase the viscosity of the solution as is described in U.S. Pat. No. 5,503,843. The t-PA may also include diluents, stabilizers, biocides, antioxidants, anti-irritants and the like.

Because of the instability of t-PA in solution, it is desirable to design transdermal patches that can be stored at room temperature. Such a dermal patch may be designed, for example, with two compartments separated by a breakable barrier; one compartment contains lyophilized t-PA and the other compartment contains a solution or carrier, such as those described above, into which the t-PA is dissolved. Prior to the use of the patch, the barrier is broken, mixing the contents of both compartments thereby forming a drug reservoir containing a solution of t-PA. Alternatively, a transdermal patch may be designed with a single breakable compartment containing lyophilized t-PA, enclosed within the liquid carrier. Prior to use of the patch, the single compartment barrier is broken releasing the lyophilized t-PA into the carrier solution. The patch is then placed in contact with the skin in such a way that the drug reservoir containing the t-PA solution is in contact with the skin.

The transdermal patch of the present invention may have a removable feature where a wearer can remove the patch even though the patch may still contain medicine to be administered. In this embodiment, the patch may have a resealing mechanism to re-seal the patch membrane to prevent any leakage of the medicine. The resealing mechanism may be in the form of a flap that may be resealed or it may be in the form of other ordinarily used resealing mechanisms. In addition, the patch adhesive for attaching the patch to the skin of a wearer may be adapted to re-adhere to a wearer's skin during reapplication of the patch to the skin. An example of a suitable adhesive is of the type used for Post-It brand sticky notes, but that are strong enough to form a secure adhesion with a patient's skin. Other adhesive mechanisms that are ordinarily used and that are within the spirit of the present invention may also be used. In a different embodiment, the patch may be designed to be removed once the reservoir of medicine has been depleted.

In addition, the use of the transdermal patch of the invention may be varied according to a physician's recommendations and a patient's needs. For example, the transdermal patch may be designed to chronically administer a dose for a specified period of time and the patch removed after the specified time period has expired. Alternatively, the dosage plan may require a new patch to be applied once the reservoir of the existing patch has been depleted to ensure a constant delivery of the thrombolytic (fibrinolytic) reagents in a therapeutically effective amount.

### Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### EXAMPLES

### Example 1

### Transdermal Administration Of Thrombolytic (Fibrinolytic) Reagents

The following example describes the administration of the thrombolytic (fibrinolytic) reagent t-PA utilizing a transdermal patch delivery system that contains an ultrasound transducer. The use of transdermal patches for the delivery of drugs through the skin is well known. Methods for the use of transdermal patches for delivery of drugs is described, for example, in the following United States patents, U.S Pat. Nos. 5,498,417, 5,503,844 and 5,503,843, each of which is incorporated by reference herein.

The following example illustrates the invention. It is not intended to limit the scope of the invention.

The t-PA (Activase, supplied by GENENTECH, Inc.) to be used in this example is supplied in 50 mg vials. The vials should be reconstituted in either sterile water or a pharmaceutical composition compatible with use in a transdermal patch.

The transdermal patch is prepared to contain a solution of t-PA and an ultrasound transducer to boost the skin's penetration by the t-PA. The t-PA is dispersed in the solution, suspension or gel in a dissolved or undissolved state. The drug reservoir of the patch containing a solution, suspension or gel of t-PA also includes permeation enhancers in addition to the ultrasound transducer on the patch. Such permeation enhancers include those described in U.S. Pat. No. 4,573,966, which is incorporated by reference herein. Permeation enhancers may include plasticizer type enhancers such as lower alky and alkoxy esters of pharmaceutically acceptable fatty acids, fatty acid esters, fatty alcohols and similar hydrophobic compounds that are capable of increasing the permeability of drugs to the skin. In addition, solvent type enhancers may be used to increase the delivery of drugs through the skin. Such enhancers generally refer to relatively hydrophilic compounds having molecular weights of less than 200. More preferably, solvent type enhancers have a molecular weight of less than 150. They are also generally greater than 2 wt % soluble in water, and are preferably greater than 10 wt % soluble in water. Typically, solvent type enhancers include pharmaceutically acceptable lower alkyl alcohol, aryl alcohol, or polyol, for example, ethanol, propanol, butanol, benzyl alcohol, glycerin, or propylene glycol. as well as diluents, such as water or other additives. The solution of t-PA may be formulated to include vascular permeability factors (VPFs), as described in U.S. Pat. No. 5,503,843, which cause a rapid and reversible increase in blood vessel permeability. Such VPF may be added to the t-PA solution to facilitate the uptake of t-PA into the blood vessels of the skin.

The amount of t-PA contained in the patch is that amount necessary to deliver a therapeutically effective dose of t-PA. The treated patient's blood is monitored to determine the levels of circulating fibrinogen and/or fibrin split products. As the t-PA is administered, the patient should be monitored to prevent excessive bleeding which can result from the treatment with thrombolytic (fibrinolytic) reagents.

Once the transdermal patch has been prepared to contain an appropriate dose of t-PA, in a suitable solution, the patient's skin is overlaid with the transdermal patch. The patch is placed in contact with the skin in such a way that the side of the patch containing the t-PA solution side is in contact with the patient's skin and the ultrasound transducer is activated. The ultrasound transducer increases the pore size of the skin for greater penetration of the t-PA into the bloodstream.

### Example 2

### Predicted T-Pa Dosage for a Once a Week Administration of T-Pa

Example 2 is an example of a once a week administration of t-PA to a patient. Example 2 in no way represents a working example. It is predicted that 5 mg of t-PA will be administered to a patient a once a week. It is further predicted that the once a week administration can continue for at least one month.

### Example 3

### Predicted T-Pa Dosage for a Twice a Week Administration of T-Pa

Example 3 is an example of a twice a week administration of t-PA to a patient. Example 3 in no way represents a working example. It is predicted that 5 mg of t-PA will be administered to a patient twice a week. It is further predicted that the twice a week administration can continue for at least one month. It is preferred that a 5 mg dose of t-PA be administered at most twice a week.

Relationship between the Amount of Thrombolytic (Fibrinolytic) Reagents Administered and the Frequency Of Administration

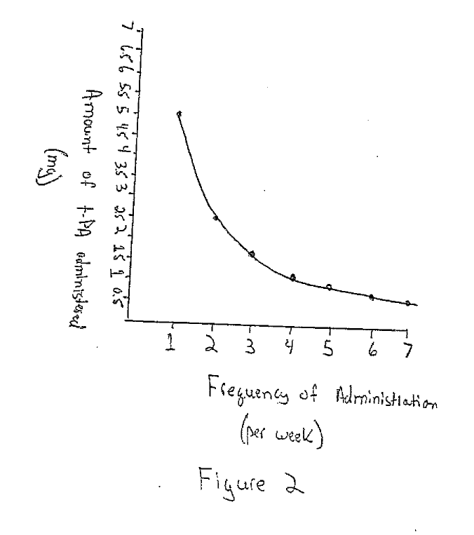

FIG. 2 is a graphical illustration of the effect that the frequency of t-PA administration will have on the amount of t-PA that is administered during each administration. The data in FIG. 2 is an estimate of therapeutically effective amounts of t-PA to be administered according to different administration frequencies and does not represent a working example. The frequency of administration in FIG. 2 varies from a weekly administration to a daily administration. FIG. 2 shows that an inverse relationship is predicted to occur between the frequency of administration and the dose of t-PA administered during each administration. It shows that as the frequency of administration is increased, that the dose of t-PA administered during each administration is predicted to decrease. The data estimated in FIG. 2 is the following: (1) for a once a week administration, about 5.0 mg of t-PA may be administered during each administration event; (2) for a twice a week administration, about 2.5 mg of t-PA may be administered; (3) for a three times a week administration, about 1.67 mg of t-PA may be administered; (4) for a four times a week administration, about 1.25 mg of t-PA may be administered; (5) for a five times a week administration, about 1.0 mg of t-PA may be administered; (6) for a six times a week administration, about 0.83 mg of t-PA may be administered; and (7) for a seven times a week administration, about 0.71 mg of t-PA may be administered.

FIG. 2 shows the predicted relationship between the amount of t-PA to be administered and the frequency of administration of t-PA per week. However, the present invention also allows for the administration of t-PA on a monthly or yearly basis and the relationship between the amount of t-PA to be administered and the frequency of administration is predicted as being substantially the same as the predicted relationship that is shown in FIG. 2.

It is predicted that therapeutically effective dosage amounts other than the amounts estimated in FIG. 2 can be used as long as the dosage is decreased as the frequency of administration is increased. A predicted range of a therapeutically effective dosage amount for a once a week administration can be from about 2.0 mg to about 10.0 mg of t-PA. A preferred predicted range would be from about 3.0 mg to about 9.0 mg of t-PA. A more preferred predicted range would be from about 4.0 mg to about 8.0 mg of a once a week t-PA administration. A most preferred predicted range would be from about 4.5 mg to about 7.0 mg of t-PA.

A predicted range of a therapeutically effective dosage amount for a twice a week administration of t-PA may be from about 1.0 mg to about 5.0 mg. Alternatively, the twice a week administration may be from about 1.5 mg to about 4.5 mg in a preferred predicted range. Even further, the twice a week administration may be from about 2.0 mg to about 4.0 mg in a more preferred predicted range. In a still further embodiment, the most preferred predicted range may be from about 2.0 mg to about 3.0 mg.

For a three times a week t-PA administration, the predicted range of a therapeutically effective dosage amount may be from about 0.7 mg to about 3.3 mg. A preferred predicted range may be from about 1.0 mg to about 3.0 mg. A more preferred predicted range may be from about 1.3 mg to about 2.7 mg. A most preferred predicted range may be from about 1.4 mg to about 2.4 mg.

A four times a week administration of t-PA may have a predicted range of a therapeutically effective dosage amount of from about 0.5 mg to about 2.5 mg. A preferred predicted range may be from about 0.75 mg to about 2.25 mg, A more preferred predicted t-PA range for a four times a week administration may be from about 1.0 mg to about 2.0 mg. A most preferred predicted range may be from about 1.0 mg to about 1.75 mg.

An administration of t-PA five times a week may have a predicted range of a therapeutically effective dosage amount of from about 0.4 mg to about 2.0 mg during each administration of t-PA. A five times a week t-PA administration preferred predicted range may be from about 0.6 mg to about 1.8 mg. A more preferred predicted range for a five times a week t-PA administration may be from about 0.8 mg to about 1.6 mg. A most preferred predicted range for t-PA administrated five times a week may be from about 0.8 mg to about 1.4 mg.

t-PA being administered six times a week may have a predicted range of a therapeutically effective dosage amount of t-PA from about 0.3 mg to about 1.7 mg. A preferred predicted range of the amount of t-PA administered during an administration may be from about 0.45 mg to about 1.55 mg. A more preferred predicted range may be from about 0.60 mg to about 1.40 mg. A most preferred predicted range of the t-PA administered may be from about 0.60 mg to about 1.25 mg.

For a seven times a week t-PA administration schedule, a predicted range of a therapeutically effective amount of t-PA to be administered may be from about 0.25 mg to about 1.40 mg of t-PA. A preferred predicted range for this schedule may be from about 0.40 mg to about 1.25 mg. A more preferred predicted administration amount range for this administration schedule may be from about 0.55 mg to about 1.10 mg. A most preferred predicted range may be from about 0.55 mg to about 0.95 mg.

The administration schedules in the present invention are not limited to the schedules that are discussed above as the administration schedule of t-PA may be once every two, three, four, five, or six or more weeks. In addition, the administration schedule may be twice every three weeks, five weeks, seven weeks, nine weeks, etc. Furthermore, the administration schedule may be calculated monthly or yearly rather than weekly and may include one administration every two months, three months, four months, five months, six months, etc. or an administration once a year, twice a year, three times a year, four times a year, five times a year, etc.

It is also important to note that the foregoing administration schedules may require the dosages to be administered equidistantly such that the time between each administration is substantially the same. In addition, the administration of the t-PA may have a constant rate of delivery regardless of the administration schedule. For example, the rate of delivery may be 0.5 mg/hr so that the administration period for the once a week administration is longer than the administration period for the twice a week administration since the once a week administration is a higher dose. Alternatively, the duration of the administration period for all of the different administration schedules may be substantially the same. In this embodiment, the rate of delivery of the doses will be adjusted so that the length of the administration period for a once a week administration substantially mirrors the length of the administration period for a different administration schedule.

The t-PA may be administered during an administration period on an hourly basis. For example, the period of administration may be one, two, three, four or more hours. The length of the administration period depends on the rate of t-PA delivery and the amount of t-PA to be delivered. For example, a rate of delivery of t-PA of 0.5 mg/hr, will make a once a week administration of 6 mg of t-PA have an administration period of 12 hours. A range for a rate of delivery of t-PA in the present invention may be from about 0.1 mg/hr to about 2 mg/hr. A preferred rate of delivery range may be from about 0.2 mg/hr to about 1.5 mg/hr. A more preferred rate of delivery range may be from about 0.3 mg/hr to about 1.0 mg/hr. A most preferred rate of delivery range may be from about 0.4 mg/hr to about 0.6 mg/hr. It is to be noted that the present invention's administration is different from currently known drug therapies that use thrombolytic (fibrinolytic) drugs for a variety of reasons. First, whereas currently known methods relate to the intravenous or intra-arterial administration of heavy doses of thrombolytic (fibrinolytic) drugs, the present invention allows for a transdermal thrombolytic (fibrinolytic) drug administration. Second, currently used therapies of thrombolytic (fibrinolytic) drugs rely on a single heavy dosage of the thrombolytic (fibrinolytic) drug. The single heavy dosage prevents a thrombolytic (fibrinolytic) drug administration on a regular basis due to hemorrhagic considerations. The present invention, on the other hand, allows for smaller dosage administrations of thrombolytic (fibrinolytic) drugs intermittently which enables the drugs to be administered more frequently. And third, because the present invention can be administered on an intermittent basis, a sustained blood level of t-PA can be obtained during a therapy. Currently known and used thrombolytic (fibrinolytic) drug therapies, on the other hand, are not able to administer a heavy dosage on a regular basis and therefore are not capable of obtaining a sustained blood level.

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims.

What is claimed is:

1) A method for treating patients suffering from a protein misfolding disease comprising:
the intermittent administration to a patient over a period of time of an effective dose of a thrombolytic reagent, said thrombolytic reagent degrading the misfolded proteins that accumulate in patients having a protein misfolding disease, said treatment maintaining circulating blood levels of from about 0.1 mg to about 50 mg of said thrombolytic reagent in a patient, and said period of time of said intermittent administration being at least one month.

2) The method according to claim 1 wherein said thrombolytic reagent is tissue plasminogen activator (t-PA).

3) The method according to claim 2 wherein said intermittent administration is a once a week administration of t-PA.

4) The method according to claim 3 wherein from about 2.0 mg to about 10.0 mg of t-PA is administered during each administration of t-PA.

5) The method according to claim 4 wherein the rate of t-PA administration is from about 0.1 mg/hr to about 2 mg/hr.

6) The method according to claim 4 wherein the rate of t-PA administration is from about 0.4 mg/hr to about 0.6 mg/hr.

7) The method according to claim 6 wherein the amount of t-PA administered is about 5.0 mg.

8) The method according to claim 7 wherein the rate of t-PA administration is 0.5 mg/hr.

9) The method according to claim 8 wherein each administration of t-PA lasts for about 10 hours.

10) The method according to claim 9 wherein the period of time is one year.

11) The method according to claim 9 wherein the period of time is six months.

12) The method according to claim 1 wherein the amount of time between each administration of said thrombolytic reagent over said period of time is substantially the same.

13) The method according to claim 2 wherein said protein misfolding disease is Alzheimer's disease.

14) The method according to claim 13 wherein said t-PA degrades beta-amyloid plaques that accumulate in the brain of a patient with Alzheimer's disease.

15) The method according to claim 1 wherein said thrombolytic reagent is administered transdermally.

16) The method according to claim 1 wherein a D-dimer test is performed before a thrombolytic reagent is administered to a patient.

17) A transdermal drug delivery system for delivering an effective dose of at least one thrombolytic reagent to a patient for the treatment of a protein misfolding disease, comprising: a transdermal patch having a rate controlling membrane with a reservoir of said thrombolytic reagent behind said rate controlling membrane and an ultrasound transducer for enhancing the penetration of said thrombolytic reagent into a patient's bloodstream by increasing the permeability of the patient's skin.

18) The transdermal drug delivery system according to claim 17 wherein said thrombolytic reagent is t-PA.

19) The transdermal drug delivery system according to claim 18 wherein the rate controlling membrane releases the t-PA at a rate of from about 0.1 mg/hr to about 2.0 mg/hr.

20) The transdermal drug delivery system according to claim 18 wherein the rate controlling membrane releases the t-PA at a rate of from about 0.4 mg/hr to about 0.6 mg/hr.

21) The transdermal drug delivery system according to claim 20 wherein the rate controlling membrane releases the t-PA at a rate of about 0.5 mg/hr.

22) A method for treating patients suffering from a protein misfolding disease comprising: intermittently administering from about 2 mg of tissue plasminogen activator (t-PA) to about 10 mg of t-PA to a patient, said thrombolytic reagent degrading the misfolded proteins that accumulate in patients having a protein misfolding disease and said treatment maintaining circulating blood levels of from about 0.1 mg to about 50 mg of said thrombolytic reagent in a patient, wherein the intermittency of said intermittent administration is a once a week administration at a rate of administration of t-PA of 0.5 mg/hr, wherein each administration of t-PA lasts for at least 10 hours, wherein the intermittent administration is continued for at least one month, and wherein a D-dimer test is performed before t-PA is administered.
